(19) United States
(12) Patent Application Publication
(10) Pub. No.: US 2024/0368577 A1
(43) Pub. Date: Nov. 7, 2024

(54) ENZYMATIC MICROCHANNEL FIBER CONTACTORS

(71) Applicant: CHEMTOR, LP, Lockhart, TX (US)

(72) Inventors: Leslie WOOD, San Marcos, TX (US); Scott D. KOHL, Maize, KS (US); William LANIER, Lake Placid, FL (US)

(21) Appl. No.: 18/740,777

(22) Filed: Jun. 12, 2024

Related U.S. Application Data
(63) Continuation of application No. PCT/US2024/027513, filed on May 2, 2024.
(60) Provisional application No. 63/499,628, filed on May 2, 2023.

(51) Int. Cl. C12N 11/04

(52) U.S. Cl. CPC C12N 11/04

(57) ABSTRACT

An immobilized enzyme fiber contactor includes a plurality of fibers disposed within a hollow conduit. The fibers have an enzyme selected from an oxidoreductase, a transferase, a hydrolase, a lyases, an isomerase, or a ligase attached thereto. The enzymes can be attached to the fibers via an anchor group and, optionally, a bifunctional crosslinker. The enzymes can be applied, stripped, and reapplied without disassembling the reactor or discarding the fibers. The immobilized enzyme fiber contractor can be used to conduct two-phase or single-phase enzymatic reactions.

### CROSS-REFERENCE TO RELATED APPLICATION

The present application is a continuation of PCT International Patent Application No. PCT/US2024/027513 filed May 2, 2024, which claims benefit of priority to U.S. Provisional Patent Application No. 63/499,628 filed May 2, 2023, entitled “Enzymic Microchannel Fiber Contactors”, the disclosure of which is hereby incorporated by reference in its entirety.

### FIELD OF THE DISCLOSURE

The present disclosure relates to enzymatic processes facilitated by a fiber contactor. More particularly, the present specification is directed to an immobilized enzyme fiber contactor and the use of the same.

### BACKGROUND

A traditional commercial procedure involving stirred enzymes is the enzymatic degumming process used for vegetable oil processing. An example of this process is shown in FIG. 1. See Jiang, X., Chang, M., Wang, X., Jin, Q., & Wang, X. (2014). A comparative study of phospholipase A1 and phospholipase C on soybean oil degumming. Journal of the American Oil Chemists' Society, 91 (12), 2125-2134; Sampaio, K. A., Zyaykina, N., Wozniak, B., Tsukamoto, J., Greyt, W. D., & Stevens, C. V. (2015). Enzymatic degumming: Degumming efficiency versus yield increase. European Journal of Lipid Science and Technology, 117 (1), 81-86; and Yang, B., Zhou, R., Yang, J. G., Wang, Y. H., & Wang, W. F. (2008). Insight into the enzymatic degumming process of soybean oil. Journal of the American Oil Chemists' Society, 85 (5), 421-425. The process includes bringing the solution to the optimal pH of the enzyme, addition of the enzyme dissolved in water followed by stirring for at least an hour. The long stir times required with added enzyme are necessary because the reaction between the enzyme and oil only occurs at the oil/water interface in cases where hydrolases are involved in the enzymatic degumming of oils. After enzymatic processing, residual phospholipids are less than 10 ppm and metals are less than 5 ppm. While the addition of enzymes can produce cleaner oils, the long stir times with high shear and continual consumption of enzymes required to do so are not ideal due to capital, enzyme cost, and high energy requirements.

Most commercial enzymes are used in a solution state in stirred tank reactors. Examples include: amyloglucosidase conversion of dextrins into glucose performed in a 13-stage stirred reactor with 24 to 48 hours of retention time to get desired conversion; alpha-amylase conversion of starch to dextrins performed in 2 to 4 staged stirred reactor system with 1 to 4 hours of retention time to get the desired conversion; beta-amylase conversion of dextrins to maltose performed in stirred tank reactors; phospholipase for the hydrolysis of phospholipids is performed in a single staged stirred reactor with 2 to 6 hours of reaction time; and biodiesel production from vegetable oil and methanol is performed with an enzyme in a stirred tank for 40 to 60 hours.

Deficiencies with stirred tank enzymatic reactions include high costs and long reaction times. As new fluid enters the stirred tank for processing, additional enzyme needs to be added and reapplication imposes an increased ingredient cost for the operation. Additionally, long to very long incubation times are often used with stirred tank reactor enzymatic systems. Long incubation time is often required because the cost of the enzyme is significant. That is, to lower continual operating expenses, the enzyme dose is minimized by increasing the reaction time. In most industrial applications, the substrate is in great excess to the enzyme, resulting in lower enzyme cost but very long reaction time requirements.

Deficiencies with packed bed immobilized enzyme reactors include high pressure drop or reduced surface area, inability to accommodate suspended solids, time and material costs for replacing beds, and channeling issues. In particular, higher surface area in a packed bed is achieved by smaller resin particle sizes, thereby leading to a high pressure drop with packed resin beds. Larger beads can be used to manage the pressure drop, but this leads to a decrease in available surface area. Additionally, packed bed reactors do not tolerate suspended solids or reactions that can produce suspended solids. These suspended solids are “filtered out” by the packed bed and even modest accumulation of suspended solids significantly compromises the system by increasing back pressure and reducing total throughput. Further, once the enzyme activity has dropped below the minimum required, the packing must be removed and thrown away thereby producing added waste. New immobilized packing needs to be purchased as replacement packing, increasing the operation costs. Lastly, channeling within the packed bed is a problem. Effective packing of the resin within the column housing is generally a complicated process requiring significant time. The effort to pack the bed, producing a uniform column with little or no channels—thus eliminating the potential for short circuits in fluid processing paths—is non-trivial.

Industrially, there are several applications where immobilized enzyme systems, using packed beds or beads in mixing tanks, impart a large cost savings given the scale of the production. A small number of examples include: glucose isomerase for the conversion of glucose to fructose (this is the largest application of immobilized enzymes in the world and is used to convert glucose from corn starch into fructose making High Fructose Corn Syrup); lipase for the Sn1.3 interesterification of fatty acids at the Sn-1 and Sn-3 position of TAG (commercially, the lipase most commonly used is isolated from Thermomyces lanuginosus); beta-galactosidase for the conversion of lactose to glucose+galactose to make lactose free cow milk and derivative products; pectinase for the production of pectin solutions in fruit processing facilities; and laccase from Pyricularia oryzae for the reduction of selective phenols in red and white wines for enhanced wine quality.

As such, there remains a need for a high throughput, cost effective system for conducting enzymatic reactions. The immobilized enzyme fiber contactor and methods of using the same described herein may provide superior throughput, reduced enzyme cost and/or improved reusability, and reduced reaction times as compare with stirred tanks and/or packed beds.

### BRIEF DESCRIPTION OF THE DRAWINGS

Various embodiments of the present disclosure will be understood more fully from the detailed description given below and from the accompanying drawings. In the drawings, like reference numbers may indicate identical or functionally similar elements. Embodiments are described in detail hereinafter with reference to the accompanying figures, in which:

FIG. 1 is a diagrammatic illustration of a process flow of stirred enzymatic degumming.

FIG. 2 is a partial cutaway diagrammatic illustration of a fiber contactor according to an embodiment of the present disclosure.

FIG. 3 is a diagrammatic illustration of a fiber contactor according to an embodiment of the present disclosure.

FIG. 4 is a diagrammatic illustration of immobilized enzymes on a fiber substrate according to an embodiment of the present disclosure.

FIG. 5 is a graph showing pressure drop results of Example 1.

FIG. 6 is a graph showing the results of Example 3.

FIG. 7 is a diagrammatic illustration of a cleavage reaction in Example 6.

FIG. 8 is a graph showing the results of Example 6.

FIG. 9 is a graph showing the results of Example 7.

FIG. 10 is a graph showing the results of Example 7.

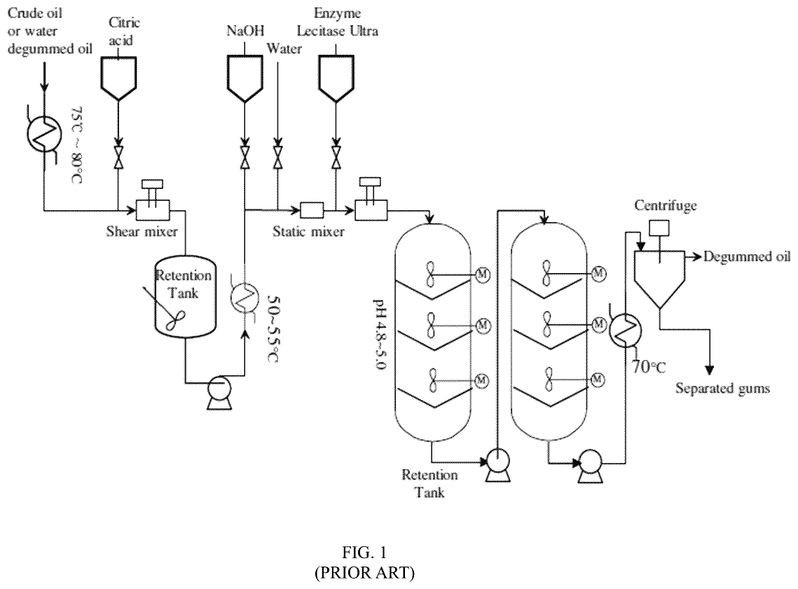

### DETAILED DESCRIPTION OF THE DISCLOSURE

The following is a description of a non-limiting method of the present disclosure used to create exemplary aerogels. Although the claimed subject matter will be described in terms of certain embodiments and examples, other embodiments and examples, including embodiments and examples that do not provide all of the benefits and features set forth herein, are also within the scope of this disclosure. Various structural, logical, and process step changes may be made without departing from the scope of the disclosure.

Ranges of values are disclosed herein. The ranges set out a lower limit value and an upper limit value. Unless otherwise stated, the ranges include all values to the magnitude of the smallest value (either lower limit or upper limit value) and ranges between the values of the stated range.

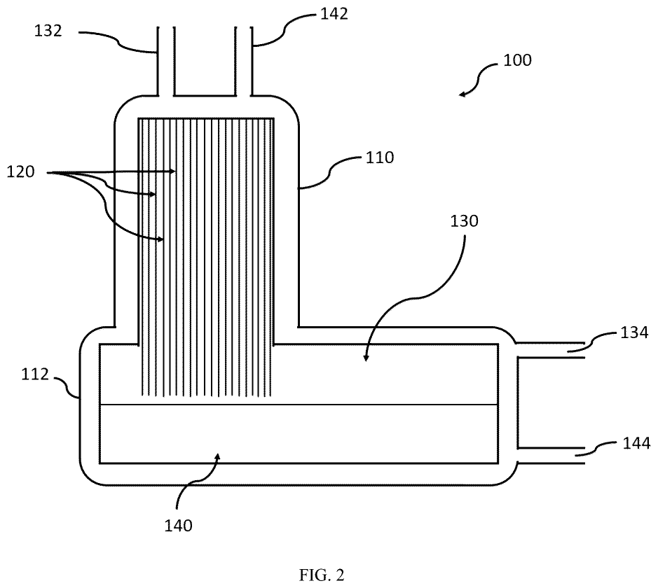

With reference to FIG. 2, the present disclosure provides a fiber contactor 100 (hereinafter an “immobilized enzyme fiber contactor” or “IEFC”) having internal elements 120 thereof (e.g., fibers) containing immobilized enzymes. The IEFC 100 includes a hollow conduit 110 housing a plurality of fibers 120. In some embodiments, the fibers 120 may be formed of steel, such as stainless steel or a steel composite. In other embodiments, the fibers 120 may be formed of basalt, ceramics, glass, polymers, and/or metals. The fibers 120 may extend an entire length of the conduit 110 or a portion thereof, or may extend beyond the conduit 110 as shown in FIG. 2. In some embodiments, the fibers 120 may be packed into the conduit 110 at a nominal rate of less than 40%, less than 30%, less than 25%, less than 20%, 5 to 30%, about 15%, or about 25%.

The conduit 110 includes one or more inlets 132, 142 for introduction of reactants into the conduit 110. In some embodiments, only a single inlet is present while other embodiments may include 2, 3, or more inlets. The IEFC 100 includes at least one outlet 134, 144 downstream of the inlet(s) 132, 142. In some embodiments, only a single outlet is present while other embodiments may include 2 or more outlets. As shown in FIG. 2, the IEFC 100 may include a separator tank or settling tank 112. In such embodiments, the separator tank 112 may include an outlet 144 for removing a denser phase 140 (e.g., an aqueous phase) therefrom and an outlet 134 for removing a lighter phase 130 (e.g., an organic phase) therefrom.

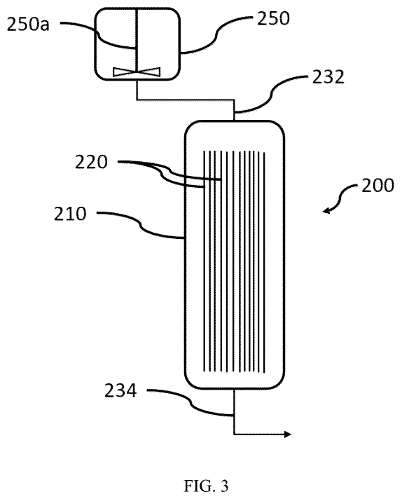

In some embodiments, the IEFC does not include a settling tank. For example, as shown in FIG. 3, an IEFC 200 includes a conduit 210 with a plurality of fibers 220 disposed therein. The IEFC includes a single inlet 232 in communication with a tank 250, which supplies a solution or mixture to the IEFC 200. The tank 250 may include a mixer 250a. The IEFC 200 also has a single outlet 234 for directing a reacted mixture from the IEFC 200. After exiting the IEFC 200, the reacted mixture may be further processed or directed to a separation process, such as a centrifuge or a drying process. Other configurations of the IEFC may be used, such as those described in U.S. Pat. No. 9,468,866 B2 by Massingill, the entirety of which is hereby incorporated by reference.

The IEFC 100, 200 enables microchannel formation along the fibers 120 to achieve high surface area to facilitate enzymatic reactions. As discussed in more detail below, such reactions may include two-phase chemistry, such as is utilized to treat phospholipids with hydrolases, Enzyme Class 3 (EC3). The IEFC 100, 200 enables high rates of interfacial bi-phasic diffusion wherein oil and water cascade down the fiber 120, 220 where intimate contact is made between the immiscible phases and the fibers 120, 220. In such embodiments, the fibers 120, 220 may extend into the separator tank 112 where the immiscible fluids disengage.

In other embodiments, the enzymatic reactions include single phase chemistry and the IEFC 100, 200 likewise provides high surface area to facilitate the enzymatic processes. Unlike hydrolases used to degum oils, which require the presence of water and a substrate oil to hydrolyze, the majority of known enzymatic reactions happen in a single phase context. Each of these enzymatic classes can equally be immobilized on the IEFC 100, 200, allowing for the reuse of enzymes across multiple batch runs.

Classes of enzymes that may be used in the present disclosure include, but are not limited to, EC1, EC2, EC3, EC4, EC5, and/or EC6. Oxidoreductases are described by enzyme class 1 (EC1) and are grouped by their functional ability to transfer electrons from one substrate to another. Another group of a commercially relevant enzymes belong to the transferases (EC2). This class of enzymes transfer functional groups from one substrate to another. These groups can be methyl-, acyl-, amino- or phosphate groups. One example, cyclodextrin glucotransferases (CGTases), have gained popularity due to their unique capacity to produce large quantities of cyclic α-(1,4)-linked oligosaccharides (cyclodextrins) from starch. These oligomers have broad utility in pharmaceutical formulations and drug delivery. Class EC3 enzymes are commonly known as hydrolases. Hydrolases add H₂O across a bond in the substrate producing two products from one substrate. These are used in many different applications such as amyloglucosidase for the conversion of dextrins to glucose, β-galactosidase for the conversion of lactose to glucose and galactose, and α-amylase for the conversion of starch to dextrins. Class EC4 enzymes are commonly known as lyases and are used in the non-hydrolytic addition or removal of groups from substrates by the modification of C—C, C—N, C—O or C—S bonds. One such example is pectinases which are commonly used to clarify fruit juices and wines. Class EC5 enzymes, isomerases, are most commonly used sugar manufacturing. Glucose isomerase (also known as xylose isomerase) catalyzes the conversion of D-xylose and D-glucose to D-xylulose and D-fructose. Like most sugar isomerases, glucose isomerase catalyzes the interconversion of aldoses and ketoses. Central to biotechnology, ligases (EC6) function to join together two molecules via the synthesis of new C—O, C—S, C—N or C—C bonds. The energy to create these bonds is provided by the simultaneous breakdown of ATP to ADP+P. On such example is the DNA ligase of the T4 bacteriophage. Functioning to assemble longer chains of DNA from shorter fragments, T4 ligase can be utilized to assemble shorter fragments of a DNA referense standard into longer segments.

In any embodiment, the IEFC 100, 200 may provide for minimal emulsion formation, an atmospherically sealed system, a small footprint as compared with traditional systems, no moving parts requiring energy and maintenance, and/or accommodation of a wide range of flow rates.

As compared with stirred tanks, the IEFC 100, 200 may solve noted issues of high costs and long reaction times. In particular, the IEFC 100, 200 allows for continual re-use of enzymes, thereby substantially reducing the operating expense of the process. Further, due to the high surface area of the fiber 120, 220, the amount of enzyme that can be immobilized within the IEFC 100, 200 can be orders of magnitude higher than can be dosed into stirred tank systems or resin beads. This much higher enzyme activity is possible because the same enzymes operate at high efficiency for months after being immobilized on the fibers 120, 220.

As compared with packed beds, the IEFC 100, 200 may solve the noted issues of high pressure drop or reduced surface area, inability to accommodate suspended solids, time and material costs for replacing beds, and/or channeling issues. In particular, the IEFC 100, 200 provides a very low pressure drop, as shown in Example 1 below, as compared to a packed resin bed reactor. Additionally, IEFC 100, 200 has channels that are more forgiving of suspended particles. Small amounts of particles will work through the IEFC 100, 200 and not build up on the system as seen with packed bed systems. Moreover, fibers 120, 220 are more tolerant of chemical treatments and can be cleaned in place with harsh treatments and enzymatic relabeling or removed and physically treated to remove extreme cases of large particulate build up while preserving the enzymatic labeled fibers. Further, once the enzyme activity within the IEFC 100, 200 is diminished, the enzymes can be removed from the column in-situ and fresh enzyme be reapplied in-situ. This eliminates the disposal of packed bed media and waste problems associated with such practices. In addition, in situations where high throughput leads to the deposition of compositions on the fibers, i.e., fouling, it is possible to treat the fibers with a clean in place (CIP) operation procedure to restore the function of the IEFC without necessitating relabeling. Lastly, the IEFC 100, 200 is robust against short circuit channeling. There is no need for slurrying of resin with liquid and subsequent packing of the media into the column housing.

The present disclosure also provides methods of immobilizing enzymes onto the internal elements of the fiber contactor as well as methods of using the IEFC to enable rapid enzymatic processing.

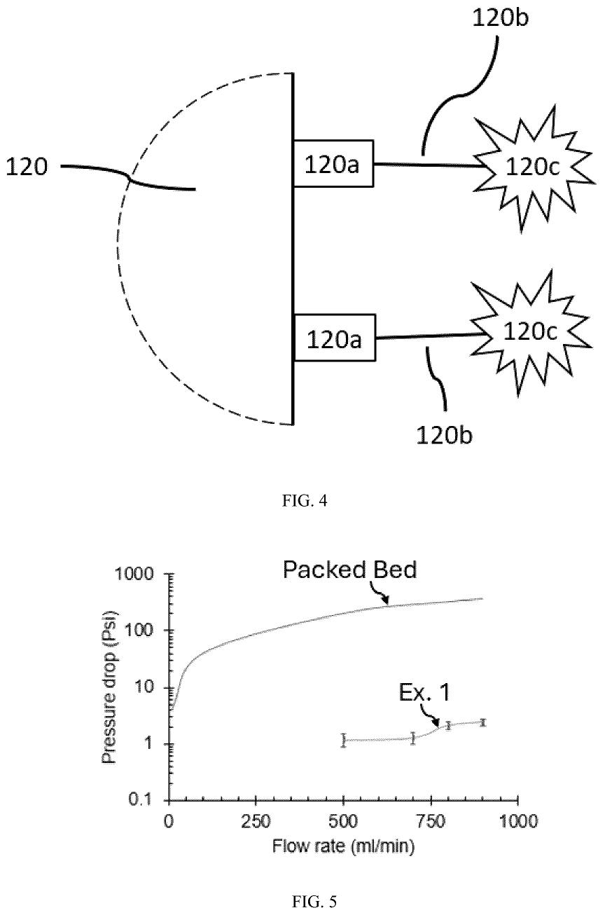

FIG. 4 depicts a fiber 120 having an enzyme 120c immobilized thereon. According to one or more embodiments, the linkage between the fiber 120 and the enzyme 120c may include a plurality of anchor groups 120a bound to the surface of the fiber 120 and a bifunctional crosslinker 120b binding the anchor groups 120a to the enzyme 120c. Many options exist for immobilizing enzymes. These methods include adsorption, covalent binding, entrapment, copolymerization and encapsulation. The general advantages and disadvantages of the various methods are summarized in Table 1 below.

TABLE 1

| | | Covalent | | | |
|---|---|---|---|---|---|
| Properties | Adsorption | Binding | Entrapment | Copolymerization | Encapsulation |
| Production | Simple | Difficult | Difficult | Simple | Simple |
| Cost | Low | High | Moderate | Cheap | Low |
| Binding | Variable | Strong | Weak | Strong | Variable |
| Force | | | | | |
| Enzyme | Yes | No | Yes | No | Yes for small |
| Leakage | | | | | molecules |
| Applicability | Wide | Selective | Wide | Wide | Wide |
| Operational | High | Low | High | Low | Low |
| Issues | | | | | |
| Matrix | Yes | Yes | Yes | No | Yes |
| Effects | | | | | |
| Considerable | No | No | Yes | No | Yes |
| Diffusional | | | | | |
| Barriers | | | | | |
| Microbial | No | No | Yes | No | No |
| Safety | | | | | |

In some embodiments, the enzyme 120c may be covalently bound to the fiber 120. In such embodiments, a process for immobilizing enzymes on internals 120 of the IEFC 100 (e.g., steel fibers) may include three steps. In the first step, the anchor groups 120a are bound to surfaces of the fibers 120. In some embodiments, the anchor groups 120a comprise an amino compound. For example, an amino silane such as aminopropyl triethoxy silane may recirculated for, e.g., 5-10 minutes against a steel fiber bundle in an ethanolic/water solution. Other amino silanes usable in the present disclosure include, but are not limited to, aminohexyl aminomethyl triethoxysilane and aminoethyl amino propyl triethoxysilane. The ethoxy silane groups undergo a hydrolysis-condensation reaction and bind to hydroxyl groups native to the surface. The same would apply to any surface with native hydroxyls (e.g., ceramic, metal, and/or glass). Once bound, the amino groups extend away from the fiber. In another example, a dopamine hydrochloride solution may be recirculated for up to 3 hours or up to 24 hours against the fibers 120 to coat the fibers 120 with polydopamine (as the anchor group 120a). The dopamine hydrochloride solution may include about 0.001 to 10 g/L or about 2 mg/L of dopamine hydrochloride and may be a buffered solution, for example using tris-HCl buffer (pH 7.4) or phosphate buffer (pH 8.5). In any embodiment, the first step may further include rinsing the fibers 120 (e.g., with water and/or denatured alcohol) to remove any unbound anchor groups 120a and drying the fibers 120 having the anchor groups 120a attached thereto. In some embodiments, drying may be conducted at elevated temperature (e.g., about 40° C. or about 80° C.) and/or under vacuum.

In the second step, an aqueous solution of a bifunctional crosslinker (e.g., glutaraldehyde) is recirculated for, e.g., about 20 minutes against the fiber bundle where the aldehyde groups crosslink with the primary amine of the amino propyl silane. Finally, an aqueous solution of enzyme is recirculated against the fiber bundle for, e.g., about 30 minutes causing the second aldehyde group on glutaraldehyde to react and covalently bond with the amine groups on the enzyme proteins. The fibers 120 may be rinsed (e.g., with deionized water) to remove unbound enzyme. All of the chemical reagents described above are inexpensive and produced at large, industrial quantities.

Other methods of immobilizing the enzymes onto the fibers may be used, as appreciated by those skilled in the art. For example, the ThermoFisher Scientific Bioconjugation technical handbook Reagents for crosslinking, immobilization, modification, biotinylation, and fluorescent labeling of proteins and peptides provides an extensive list of available linker molecules for the linkage of different functional groups. Table 2 below summarizes some of the linkers useful in the present system, method, and apparatus. In particular, Table 2 lists 21 chemicals for amine-to-amine linkages; 7 chemicals for sulfhydryl-to-sulfhydryl linkages; 28 chemicals for amino-to-sulfhydryl linkages; 3 chemicals for carboxyl-to-amine; 6 chemicals for sulfhydryl-to-carboxyl; 11 chemicals for photoreactive linkages; and 12 chemicals for chemoselective linkages.

TABLE 2
Bioconjugation chemicals available from ThermoFisher Scientific.

| | | |
|---|---|---|
| | | Number of |
| | | different |
| | | chemical |
| | | linkers |
| Functional groups linked | Chemical linker example | available |

Homobifunctional crosslinker

| | | |
|---|---|---|
| Amine-to-amine reactive (NHS | DSG (disuccinimidyl glutarate) | 11 |
| ester | | |
| Amine-to-amine reactive (NHS | BS2 G-d0 (bis(sulfosuccinimidyl) glutarate-d0) | 6 |
| ester), deuterated or MS-cleavable | | |
| Amine-to-amine reactive | DMP (dimethyl pimelimidate) | 4 |
| (imidoester or difluoro) | | |
| Sulfhydryl-to-sulfhydryl reactive | BMOE (bismaleimidoethane) | 7 |
| (maleimide) | | |

Heterobifunctional crosslinkers

| | | |
|---|---|---|
| Amine-to-sulfhydryl reactive | SIA (succinimidyl iodoacetate) | 4 |
| (NHS-haloacetyl) | | |
| Amine-to-sulfhydryl reactive | AMAS (N-α-maleimidoacetoxysuccinimide ester) | 22 |
| (NHS-maleimide) | | |
| Amine-to-sulfhydryl reactive | SPDP (succinimidyl 3-(2-pyridyldithio) propionate) | 6 |
| (NHS-pyridyldithiol) | | |
| Carboxyl-to-amine reactive | EDC (1-ethyl-3-(3-dimethylaminopropyl)carbodiimide | 3 |
| (carbodiimide plus NHS ester) | hydrochloride | |
| Sulfhydryl-to-carbohydrate or - | BMPH (N-β-maleimidopropionic acid hydrazide | 6 |
| carboxyl (maleimide, | | |
| pyridyldithiol/hydrazide, or | | |
| isocyanate | | |
| Photoreactive (NHS ester and aryl | Sulfo-SANPAH (sulfosuccinimidyl 6-(4′-azido-2′- | 9 |
| azide, phenyl azide, diazirine, or | nitrophenylamino) hexanoate) | |
| psoralen) | | |
| Chemoselective ligation (NHS ester | NHS-Azide | 6 |
| and azide-phosphine or -alkyne) | | |
| Chemoselective ligation | GlcNAz (N-azidoacetylglucosamine, tetraacylated) | 6 |
| Photoreactive amino acids | L-Photo-Leucine | 2 |

Biotin and desthiobiotin labeling reagents

| | | |
|---|---|---|
| Amine-reactive | EZ-Link NHS-Biotin | 12 |
| Sulfhydryl-reactive | EZ-Link BMCC-Biotin | 4 |

Modification reagents for reduction and denaturation of proteins

| | | |
|---|---|---|
| Disulfide bond reduction | 2-Mercaptoethanol/(β-mercaptoethanol) | 6 |
| Schiff base reduction to alkylamine | AminoLink Reductant (sodium cyanoborohydride) | 1 |
| linkage | | |
| Protein denaturants and chaotropes | Guanidine-HCl | 2 |

Modification reagents for proteins and peptides

| | | |
|---|---|---|
| Irreversibly blocks primary amines | Pierce Sulfo-NHS-Acetate (sulfo-N- | 1 |
| | hydroxysulfosuccinimide acetate | |
| Modifies primary amines to contain | Pierce SATP (N-succinimidyl S-acetylthio-propionate) | 3 |
| a protected sulfhydryl group | | |
| Modifies primary amines to contain | Traut's Reagent (2-iminothiolane) | 1 |
| a free sulfhydryl group | | |
| Adds amine or carboxylic acid | Pierce AEDP (3-((2-aminoethyl)dithio) propionic acid- | 1 |
| functional group to protein or | HCl) | |
| surface | | |
| Irreversibly blocks sulfhydryl | Pierce NEM (N-ethylmaleimide) | 1 |
| groups | | |
| Reversibly blocks sulfhydryl groups | Pierce MMTS (methyl methanethiosulfonate) | 1 |
| Adds primary amine to glass and | Pierce APTS (3-aminopropyltriethoxysilane | 1 |
| silica surfaces through silylation | | |
| Oxidizes carbohydrates for | Pierce Sodium Meta-Periodate | 1 |
| reductive amination | | |
| Alkylates reduced cysteines | Pierce Iodoacetic Acid | 3 |
| Deprotects SATA-modified | Pierce Hydroxylamine-HCl | 1 |
| molecules | | |

PEGylation (PEG labeling) reagents for proteins

| | | |
|---|---|---|
| Amine-reactive linear PEGylation | MS(PEG)4 (methyl-PEG4-NHS ester) | 4 |
| of protein or surface, terminating | | |
| with a methyl group | | |
| Amine-reactive branched | TMS(PEG)12 ((methyl-PEG12) 3-PEG4-NHS ester | 1 |
| PEGylation of a protein or surface, | | |
| terminating with a methyl group | | |
| Sulfhydryl-reactive branched | MM(PEG)12 (methyl-PEG12-maleimide) | 2 |
| PEGylation of a protein or surface, | | |
| terminating with a methyl group | | |
| PEGylation of a protein or surface, | CA(PEG)4 (carboxyl-(4-ethyleneglycol) ethylamine | 4 |
| terminating with a carboxylic acid | | |
| or primary amine | | |
| PEGylation of a gold, silver, or | CL(PEG)12 Carboxy-PEG-Lipoamide Compound | 3 |
| metal surface, terminating with a | | |
| carboxylic acid or methyl group | | |
| PEGylation of a protein or inert | MT(PEG)4 Methyl-PEG-Thiol Compound | 1 |
| material surface, terminating with a | | |
| methyl group | | |
| PEGylation of a protein, oxidized | CL(PEG)12 Carboxy-PEG-Lipoamide Compound | 3 |
| carbohydrate, or surface, | | |
| terminating with a methyl group | | |
| PEGylation of a protein or inert | MT(PEG)4 Methyl-PEG-Thiol Compound | 1 |
| material surface, terminating with a | | |
| methyl group | | |
| PEGylation of a protein, oxidized | MA(PEG)4 (methyl-(4-ethyleneglycol) ethylamine) | 4 |
| carbohydrate, or surface, | | |
| terminating with a methyl group | | |

Fluorescent dye labeling reagents and kits

| | | |
|---|---|---|
| Amine-reactive | Alexa Fluor 350 NHS Ester (Succinimidyl Ester) | 65 |
| Sulfhydryl-reactive | Alexa Fluor 350 C5 Maleimide | 18 |
| Carboxyl-reactive | Alexa Fluor 350 Hydrazide | 20 |
| Chemoselective | Alexa Fluor 488 Azide | 28 |

In some embodiments, the enzyme 120c may be bound without using the bifunctional crosslinker 120b. For example, the enzyme 120c may be bound to the fiber 120 using Van der Waals forces. In such embodiments, the fibers 120 may first be cleaned of any oils or organic compounds by circulating a suitable organic solvent such as hexane or denatured alcohol or ethanol against the fibers 120 for a period of time, for example, about 20 minutes. After the fibers 120 are cleaned the solvent may be rinsed to remove non-polar organic solvent. A 95/5 (by volume) ethanol (or denatured alcohol) and water solution is acidified to a pH of about 4.5 with an acid such as acetic acid. The acid may act to accelerate hydrolysis-condensation reactions. To this mixture, about 1 to 5 vol % of trimethoxyhexadecyl (or octadecyl or octyl) silane is added, and the resultant mixture is recirculated for a period of time (e.g., about 10 minutes) over the fibers 120. The fluid is drained and the fibers 120 are rinsed with denatured alcohol to remove loosely bound alkyl silane. The fibers may then be dried (e.g., at about 110° C. for about 10 minutes) to drive silane crosslinking to completion. The enzyme 120c may then be circulated over the alkyl functionalized fibers for about 20-30 minutes to bind the enzyme 120c to the fibers 120. The fibers 120 may be rinsed with DI water to remove any unbound enzyme. In some embodiments, the enzyme 120c may be PLA or PLC and circulating the enzyme 120c over the fibers 120 may be accomplished using a mixture of 1 ml of enzyme 120c per 1 L of water.

In some embodiments, the enzymatic reactions described herein may be conducted at room temperature or an elevated temperature of, for example, 30-80° C., 40-70° C., 50-60° C., 50-55° C., about 50° C., about 55° C., or about 60° C. Depending on the enzyme used, the temperature may be appropriately adjusted to an optimal temperature range thereof. In some embodiments, the reaction is conducted at a reduced pH of less than 7, less than 6, about 4 to 6, about 4 to 5, about 4, about 4.5, or about 5. As with the temperature, the pH may be controlled to an optimal range for the enzyme or enzymes used in the IEFC.

In any embodiment, a pressure within the IEFC may be maintained at about 1 to 125 psi, about 1 to 100 psi, about 1 to 50 psi, or about 20 psi. In some embodiments, a total flow rate of the reactants per cross-sectional area (cm²) of the IEFC may be at least 10 ml/min/cm², at least 15 ml/min/cm², at least 25 ml/min/cm², at least 50 ml/min/cm², at least 75 ml/min/cm², at least 100 ml/min/cm², at least 125 ml/min/cm², at least 150 ml/min/cm², or 20 to 250 ml/min/cm². The IEFC may be readily scaled to accommodate industrial quantities. Moreover, the contact time needed to effectuate the reactions described herein is drastically reduced as compared to traditional methods.

The present disclosure also provides for methods of treating solutions and/or oils with the IEFC. In such methods, the solution and/or oil includes a reactant capable of undergoing an enzymatic reaction in the presence of the enzyme immobilized on fibers of the IEFC. The method includes introducing the solution and/or oil into the IEFC and contacting it with the fibers and the enzyme immobilized thereon and then receiving a product solution or oil from an outlet of the IEFC, wherein the reactant has been chemically modified in the product solution or oil. In some embodiments, the solution or oil is a solution comprising a reactant dissolved in a solvent, such as water. In some embodiments, the enzyme is an oxidoreductase and the method includes removing one or more hydrogen atoms from the reactant. In some embodiments, the enzyme is a transferase, the solution comprises a donor reactant comprising a functional group (e.g., a methyl group) and an acceptor reactor, and the method includes transferring the functional group from the donor reactant to the acceptor reactant. In some embodiments, the enzyme is a hydrolase and the method includes reacting the reactant with water to cleave one or more bonds of the reactant. In some embodiments, the enzyme is a lyases and the method includes cleaving one or more bonds of the reactant by means other than hydrolysis or oxidation. In some embodiments, the enzyme is an isomerase and the method includes isomerizing the reactant. In some embodiments, the enzyme is a ligase, the solution comprises two reactants which may be the same or different, and the method includes forming a bond between the two reactants.

### EXAMPLES

### Example 1

Pressure drop was measured for a packed column with 50-micron silica beads and a 34% packed fiber contactor including 50-micron fibers. The packed column and the fiber contactor had the same pack rate (34%), height, and fluid conditions (temperature, viscosity, and flow rate). The results are shown in FIG. 2.

### Example 2: Immobilization of Enzyme onto Fibers

77 g of 50-micron stainless steel crimped fibers measuring 1 foot long were rinsed with denatured alcohol to remove any oils/organics that are found on the steel fiber for approximately 10 minutes. A solution of 2% (3-aminopropyl) triethoxysilane (APTES), 5% water, and balance denatured alcohol was recirculated across the fibers for 10 minutes and then drained. The fibers were then rinsed for another 10 minutes with fresh denatured alcohol, drained, and then cured at 100° C. in an oven overnight. A 500 ml 0.5% glutaraldehyde solution in water was recirculated over the fibers for 1 hour and then the fibers were rinsed for 10 minutes with fresh water. The fibers were positioned in a glass column of a fiber contactor having a 0.5″ inner diameter and 1 foot length, the fibers accounted for 25% of the reactor column's void space. An enzyme solution was recirculated over the fibers for 2 hours followed by a static hold inside the column for 4 nights. The enzyme solution was then recirculated for 24 hours before being rinsed for 10 minutes with fresh water to remove loose enzyme. Optimal flow rates and fiber packing ratios exist for each enzymatic process in which the parameters of residence time, internal mixing (laminar vs turbulent) and enzyme concentration (modulated through packed fiber surface area) are to be considered. For the sake of simplicity, when not described, the following examples were performed in a reactor having a 25% pack of 50-micron fibers and held for described periods of time prior to samples being taken.

### Example 3: Glucose Oxidase (EC1)

The process of Example 2 was followed using an enzyme solution of 1 gram of powdered glucose oxidase enzyme dissolved in 500 ml water. The column was held at 50° C. to which 50 ml of a 30 mM glucose solution in pH 5.7 buffer was recirculated for 1 hour. The buffer was made by mixing 175 ml of 0.1M citric acid and 500 ml of 0.2M Na₂HPO₄.

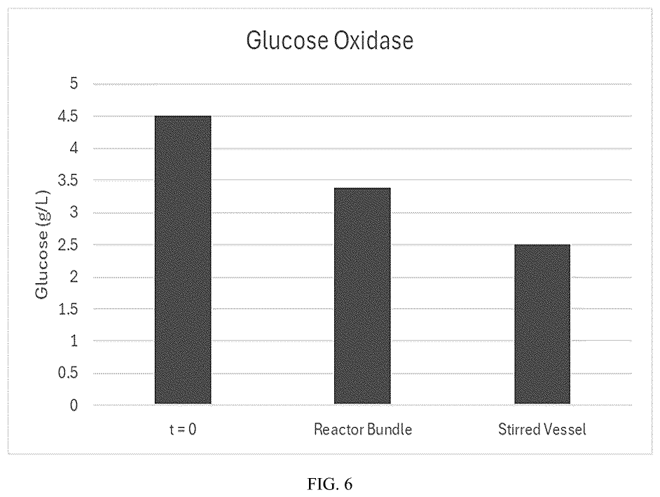

As a stirred vessel comparison, 1 g of glucose oxidase enzyme powder was added to 50 ml of the 30 mM glucose solution in pH5.7 buffer. This was stirred for 1 hr at 50° C. at 250 rpm. The results are shown in FIG. 6. The immobilized contactor bundle and the stirred vessel are graphed against the 30 mM solution (t=0) of the stirred vessel before any reaction occurs for a baseline comparison.

The above example evaluates the utility of glucose oxidase (EC1 group enzymes) in the fiber contactor to generate hydrogen peroxide using glucose as the starting substrate. This application could be used in yeast fermentation processes. In this process, yeast are more resistant to H₂O₂ than competing bacteria contamination. In fact, the yeast often can use small amounts of H₂O₂ as a growth factor when the molecule decomposes to oxygen and water. The fermentation process uses glucose as the main substrate thus there will be large quantities of low-cost glucose present for raw material as the substrate to produce the H₂O₂. The H₂O₂ will then preferentially kill bacterial species (Lactobacillus in particular) allowing the yeast fermentation to proceed with higher efficiency.

### Example 4: Glucoamylase Enzyme (EC3)

Glucoamylase enzyme was immobilized on a fiber contactor as in Example 2. Store-bought maltodextrin of unknown chain length and degree of branching was dissolved in a 0.1M citrate buffer at pH 4.5 and a concentration of 0.17 grams per milliliter. This solution was flowed over the fiber contactor in one, two, three, four, five and six passes at 50° C. and recirculated for 16 hours at room temperature. Each transit through the fiber contactor took less than 1 minute. Glucose increase was tracked with Brix meter and glucose test strips. The results are summarized in Table 3 below.

TABLE 3

| Number of Passes Maltodextrin | |
|---|---|
| Solution Through Glucoamylase | |
| Immobilized Fiber Reactor | Brix Meter Measurement Average of 4 |
| Column | Measurements |
| 1 | 14.82% |
| 2 | 15.0% |
| 3 | 15.3% |
| 4 | 15.5% |
| 5 | 15.75% |
| 6 | 16.25% |
| 192 (16 hours recirculation) | 24.17% |

The glucose test strips likewise showed an increase in the glucose concentration.

### Example 5: Lactase Enzyme (EC3)

Lactase enzyme was immobilized on a fiber contactor as in Example 2. Store-bought skim milk (pH 6.8) was flowed over the fiber contactor in one, two, and three passes and recirculated for 14 hours at room temperature. Each transit through the fiber contactor took less than 1 minute. Glucose increase was tracked with Brix meter and glucose test strips. The results are summarized in Table 4 below.

TABLE 4

| Sample | Glucose Concentration | |
|---|---|---|
| Crude | 0 | mg/dL |
| 1^{st} pass | 100-300 | mg/dL |
| 2^{nd} pass | 300 | mg/dL |
| 3^{rd} pass | 1000 | mg/dL |
| Overnight (14 hours | 1000-3000 | mg/dL |

recirculation)

The glucose test strips likewise showed an increase in the glucose concentration. After 3 passes, the glucose level was ˜1 w/v % and overnight the glucose level was approaching 3 w/v %. The theoretical yield of glucose would be 2.63 w/v % so it appears the fiber bound lactase had good activity even at room temperature.

### Example 6: Pectinase (EC4)

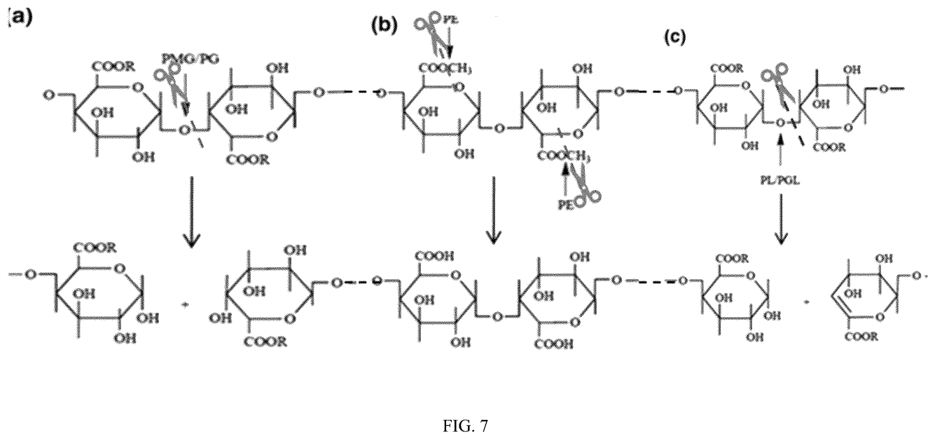

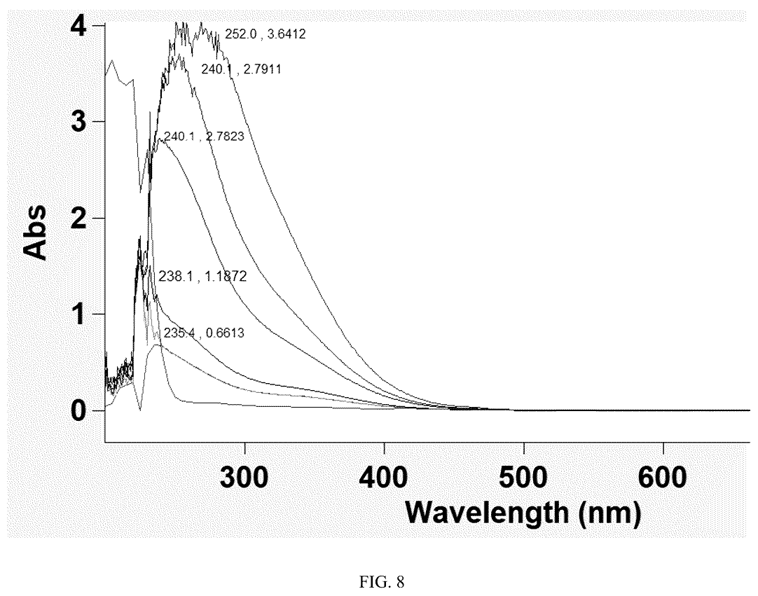

Fruit pectin (available under the trademark SURE JELL) was used to make a 2 L solution consisting of pectin at 0.5 w/v % in a 50 mM citrate buffer at pH 4. Pectinase was immobilized in the fiber contactor as in Example 2 and the fiber contactor was warmed through a condenser jacket with heated water at 60° C. The pectin solution was passed through the column at 25 ml/min for 2 minutes and then the pump was paused, and the column was sealed with the pectin solution inside. After 1 minute, the solution was emptied and collected. The fiber column was rinsed with fresh pectin solution and then sealed and held for 5 minutes before emptying and collecting the solution. The procedure was repeated several more times to produce a several more samples at different residence times on the fiber. The samples consisted of 1 minute, 5 minutes, 10 minutes, 30 minutes, and 60 minutes. All of the reactions were performed at 60° C. When pectin enzyme breaks down the polysaccharide bonds as shown in FIG. 7, an unsaturated bond forms on one of the resulting rings. This formation of the unsaturated uronide molecule is monitored by an increase in absorption at 235 nm. FIG. 8 displays the results. At 300 nm, the bottom line is a baseline measurement (0.5 wv % solution of pectin in 50 mM citrate buffer at pH4), the next line up is the 1-minute sample (0.66 abs at 235 nm), the next line up is the 5-minute sample (1.18 abs at 238 nm), the next line up is the 10-minute sample (2.78 abs at 240 nm), the next line up is the 60-minute sample (2.79 abs at 240 nm), and the top line is the 30-minute sample (3.64 abs at 252 nm).

### Example 7: Glucose 6P Isomerase (EC5)

The procedures of Example 2 were followed and, as the enzyme solution, a 0.4 ml of glucose 6P isomerase from baker's yeast was dissolved in 500 ml of water and recirculated over the bundle for 1 hour and then rinsed with fresh water for 10 minutes.

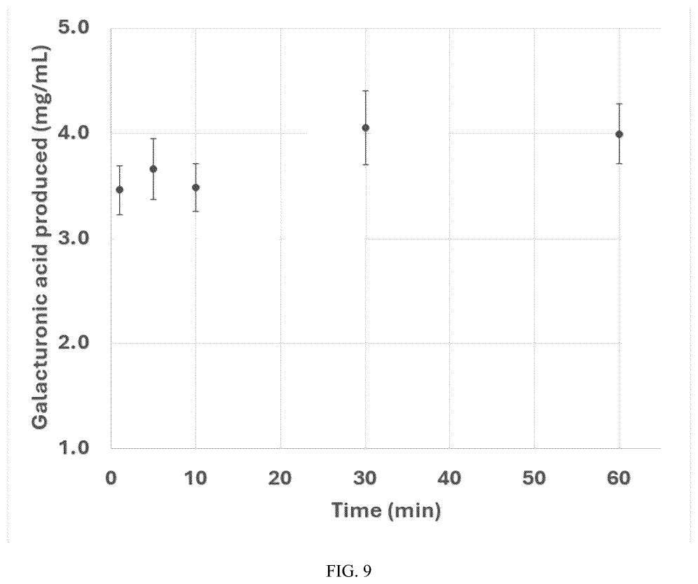

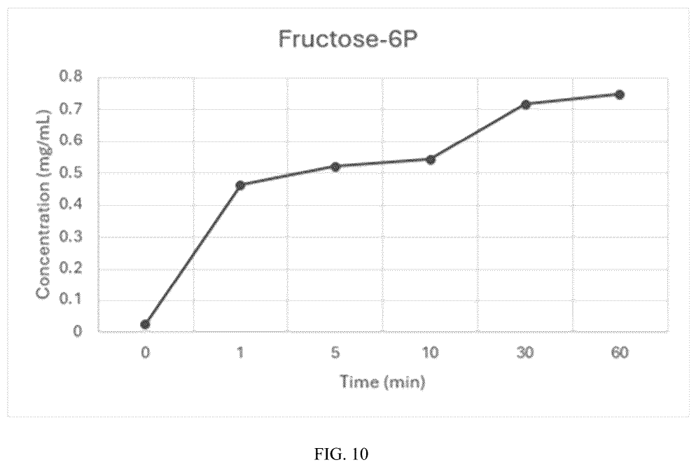

A buffer solution was made by mixing 0.2M Na₂HPO₄ with 0.1M citric acid at a ratio of ˜14.7 to 1 respectively to produce a pH of 7.6. To this buffer, glucose phosphate was added to make a 10 g/L or 38 mM concentration. The glucose phosphate solution was held in the fiber contactor for different residence times at 50° C. The column was filled with the solution, the timer was started and then the solution was removed before filling with fresh solution to begin a new residence time trial. Residence times tested included: 1, 5, 10, 30 and 60 minutes. FIGS. 9 and 10 show the results of the trials.

Although several embodiments have been disclosed in detail above, the embodiments disclosed are not limiting, and those skilled in the art will readily appreciate that many other modifications, changes, and substitutions are possible in the disclosed embodiments without materially departing from the novel teachings and advantages of the present disclosure. Accordingly, all such modifications, changes, and substitutions are intended to be included within the scope of this disclosure as defined in the following claims. In the claims, any means-plus-function clauses are intended to cover the structures described herein as performing the recited function and not only structural equivalents, but also equivalent structures. Moreover, it is the express intention of the applicant not to invoke 35 U.S.C. § 112 (f) for any limitations of any of the claims herein, except for those in which the claim expressly uses the word “means” together with an associated function.

What is claimed is:

1. A method comprising:
introducing a solution comprising a reactant dissolved in a solvent into a microchannel contactor;
wherein the microchannel contactor comprises a hollow conduit having a plurality of fibers disposed therein,
wherein the plurality of fibers form microchannels therebetween; and
wherein the plurality of fibers comprise an enzyme immobilized on surfaces thereof, the enzyme selected from an oxidoreductase, a transferase, a hydrolase, a lyases, an isomerase, or a ligase;
enzymatically reacting the reactant within the microchannels to chemically modify the reactant; and
collecting the solution comprising the chemically modified reactant from the microchannel contactor.

2. The method of claim 1, wherein the enzyme comprises an oxidoreductase and the reactant comprises a hydrogen atom and the method comprises removing the hydrogen atom from the reactant.

3. The method of claim 1, wherein the enzyme comprises a transferase, the reactant comprises a donor reactant comprising a functional group and an acceptor reactor, and the method comprises transferring the functional group from the donor reactant to the acceptor reactant.

4. The method of claim 1, wherein the enzyme comprises a hydrolase and the method comprises reacting the reactant with water to cleave one or more bonds of the reactant.

5. The method of claim 1, wherein the enzyme comprises a lyases and the method comprises cleaving one or more bonds of the reactant.

6. The method of claim 1, wherein the enzyme comprises an isomerase and the method comprises isomerizing the reactant.

7. The method of claim 1, wherein the enzyme comprises a ligase, the reactant comprises a first reactant and a second reactant, and the method comprises forming a bond between the first reactant and the second reactant.

8. A method comprising:
providing a hollow conduit;
disposing a plurality of fibers within the hollow conduit, wherein the fibers comprise hydroxyl groups on surface thereof;
contacting an anchor group precursor in an ethanolic/water solution with the fibers to bind an anchor group to the hydroxyl groups of the fibers;
contacting an aqueous solution of a bifunctional crosslinker with the anchor group bound to the fibers; and
contacting an aqueous solution of enzyme with the fibers to bind the enzyme to the fibers, the enzyme selected from an oxidoreductase, a transferase, a hydrolase, a lyases, an isomerase, or a ligase.

9. The method of claim 8, wherein the enzyme comprises an oxidoreductase.

10. The method of claim 8, wherein the enzyme comprises a transferase.

11. The method of claim 8, wherein the enzyme comprises a hydrolase.

12. The method of claim 8, wherein the enzyme comprises a lyases.

13. The method of claim 8, wherein the enzyme comprises an isomerase.

14. The method of claim 8, wherein the enzyme comprises a ligase.

15. The method of claim 8, wherein the anchor group precursor is aminopropyl triethoxy silane, aminohexyl aminomethyl triethoxysilane, aminoethyl amino propyl triethoxysilane, or dopamine hydrochloride and the bifunctional crosslinker is glutaraldehyde.

16. The method of claim 8, further comprising removing the enzyme from the fibers by simultaneously introducing a strong base and an alcohol or a solution of H₂O₂ and sulfuric acid into the hollow conduit.

17. The method of claim 16, further comprising reapplying the enzyme by repeating the contacting an anchor group precursor, the contacting an aqueous solution of a bifunctional crosslinker, and the contacting an aqueous solution of enzyme.

18. An apparatus comprising:
a hollow conduit having an inlet, an outlet, and a plurality of fibers disposed therein;
wherein the plurality of fibers form microchannels therebetween;
wherein the plurality of fibers comprise an enzyme immobilized on surfaces thereof, the enzyme selected from an oxidoreductase, a transferase, a hydrolase, a lyases, an isomerase, or a ligase; and
wherein the enzyme is immobilized on the plurality of fibers via a silane compound.

19. The apparatus of claim 18, further comprising a mixing tank comprising a mixing implement, the mixing tank being fluidically coupled to the inlet.

20. The apparatus of claim 18, wherein the silane compound is an amino silane or polydopamine and the enzyme is covalently bound to the silane compound via glutaraldehyde.
